(19) United States
(12) Patent Application Publication
(10) Pub. No.: US 2025/0140424 A1
(43) Pub. Date: May 1, 2025

(54) CELL THERAPY PROTOCOL SYSTEMS AND METHODS

(71) Applicant: Elixirgen Therapeutics, Inc., Baltimore, MD (US)

(72) Inventors: Akihiro KO, Baltimore, MD (US); Minoru S.H. KO, Cockeysville, MD (US)

(73) Assignee: Elixirgen Therapeutics, Inc., Baltimore, MD (US)

(21) Appl. No.: 18/835,667

(22) PCT Filed: Feb. 3, 2023
(86) PCT No.: PCT/US2023/012300

§ 371 Date: Aug. 2, 2024

Related U.S. Application Data
(60) Provisional application No. 63/306,711, filed on Feb. 4, 2022.

(51) Int. Cl. G16H 50/70; G16B 20/00; G16B 30/00; G16H 10/60; G16H 20/10

(52) U.S. Cl. CPC G16H 50/70; G16B 20/00; G16B 30/00; G16H 10/60; G16H 20/10

(57) ABSTRACT

Disclosed herein are systems and methods for cell therapy treatment or training in a cell therapy suite. The suite can receive information that includes a library of cell therapy protocols for a plurality of combinations of patient indications and genes of interest (GOI). The suite can receive a request for treating a patient with cell therapy based on a combination of the patient's indication and GOI and in response to receiving the request, perform a step of a respective cell therapy patient protocol from the library of cell therapy protocols that corresponds to the combination of the patient's indication and GOI.

### CROSS-REFERENCE TO RELATED APPLICATIONS

This application claims the benefit of U.S. Provisional Application No. 63/306,711 filed Feb. 4, 2022, the entire contents of which are incorporated herein by reference.

### FIELD

This relates generally to a systems and methods for performing cell therapy on patients, and more specifically to systems, methods, and suites for providing, performing, and maintaining the most up to date operating conditions and protocols for cell therapy.

### BACKGROUND

There are many issues with bringing cell therapy to the public. First, unless the treatment is for an indication with a large or close-to universal market size, there is not enough potential volume of business for creating a cell therapy treatment system for a single indication, especially given that therapies may become approved for subsets of their initial targets, which would reduce their market size. Second, the high specialization and specificity per-treatment required in the planning, management, and execution of the deployment of a commercial cell therapy, as well as the high skill necessary for the technical workforce, disincentivizes the creation or use of a common platform. Finally, since the field of cell and gene therapy is still in an early stage, any common platforms that are created (which require a huge early investment) could become quickly usurped by the ongoing development of new technologies.

### SUMMARY

To enable widespread use and adoption of cell therapy, the treatment system should be based on a platform with large market sizes, be usable as a common platform for more than one indication, be scalable in deployment and management, and be an adaptable platform that can incorporate newer technologies while retaining its original or existing features. Applicants have discovered such systems and methods that can employ a suite for facilitating cell therapy to patients. In addition, Applicants have discovered systems and methods for training the technical workforce involved with delivering cell therapy to patients.

Each cell therapy is different: current cell therapies can have differing protocols, and are often adjusted for scalable production to match the protocol. For example, CAR-T cell therapy's protocol has been optimized for sending the patient cells to a centralized location, treating them, and then sending them back for infusion. However, the systems and methods disclosed herein can employ a local-only approach, and can avoid some of the customizations required for such workflows. In addition, since the treatment and culture can be automated and software-based in the suites, new protocols and changes to protocols can be updated and “patched” via a network connection in real time. Such updates could also incorporate new findings and new technologies or protocol optimizations, ensuring that the cell therapy remains up-to-date with the latest information.

The suites disclosed herein can be a compact, modular, standalone, and/or scalable common platform for cell therapies with applicability to virtually any cell therapy, and the flexibility to do so rapidly. Since the suites disclosed herein are compact, modular, and also standalone, they can be deployed in hospitals and clinics with a very small footprint. As such, the mini-Good Manufacturing Practice (GMP) suites can be placed in virtually any room with the only requirement being the floor space (and external power in some situations). These suites can also be placed in hard-to-reach areas as long as initial supply of reagents and consumable (e.g., tubing, sterile bags, other plasticware, etc.) is sufficient. In addition, any data gathered from performing cell therapy treatments in the suites by the overall system can be used to make treatments even better or customizable. Finally, the systems and methods (and suites) disclosed herein can lead to reducing costs overall for all cell therapies through economies of scale and efficiencies gained through a local-only strategy.

In some embodiments, a cell therapy suite includes one or more processors configured to receive information comprising a library of cell therapy protocols for a plurality of combinations of patient indications and genes of interest (GOI); receive a request for treating a patient with cell therapy based on a combination of the patient's indication and GOI; and in response to receiving the request for treating a patient with cell therapy, perform a step of a respective cell therapy patient protocol from the library of cell therapy protocols that corresponds to the combination of the patient's indication and GOI. In some embodiments, the received information is from a remote server location outside of the suite. In some embodiments, the suite comprises a cell-processing device. In some embodiments, the information comprising the library of cell therapy protocols comprises a plurality of operating conditions for the cell-processing device for the plurality of combinations of patient indications and GOIs. In some embodiments, receiving a request for treating a patient with cell therapy based on a combination of the patient's indication and GOI comprises receiving a request for an operating condition of the cell-processing device based on a combination of the patient's indication and GOI. In some embodiments, in response to receiving the request for the operating condition, the one or more processors are configured to process cells of the patient with RNA carrying the GOI in the cell-processing device according to a respective operating condition that corresponds to the combination of the patient's indication and GOI. In some embodiments, the RNA is c-srRNA. In some embodiments, the one or more processors are configured to receive modifications to the library of cell therapy protocols for a plurality of combinations of patient indications and GOIs. In some embodiments, the cell therapy suite is a clean room that satisfies ISO 5, ISO 7, or ISO 8. In some embodiments, the suite includes an RNA production unit configured to load GOIs onto an RNA vehicle. In some embodiments, the one or more processors are configured to receive a patient's GOI identified by a sequencer and in response to receiving the patient's identified GOI, load the identified GOI onto an RNA vehicle in the RNA production unit or the one or more processors are configured to transmit the identified GOI information to a remote server location outside of the suite. In some embodiments, the suite includes a sequencer to identify the patient's GOI. In some embodiments, the suite includes an apheresis device for extracting the patient's cells.

In some embodiments, a system for cell therapy treatment includes a cell therapy suite; and one or more processors configured to: receive information comprising a library of cell therapy protocols for a plurality of combinations of patient indications and genes of interest (GOI); receive a request for treating a patient with cell therapy based on a combination of the patient's indication and GOI; and in response to receiving the request for treating a patient with cell therapy, perform a step of a respective cell therapy patient protocol from the library of cell therapy protocols that corresponds to the combination of the patient's indication and GOI.

In some embodiments, a method performed at a system comprising a cell therapy suite and one or more processors, the method includes: receiving information comprising a library of cell therapy protocols for a plurality of combinations of patient indications and genes of interest (GOI); receiving a request for treating a patient with cell therapy based on a combination of the patient's indication and GOI; and in response to receiving the request for treating a patient with cell therapy, performing a step of a respective cell therapy patient protocol from the library of cell therapy protocols that corresponds to the combination of the patient's indication and GOI.

In some embodiments, a non-transitory computer-readable medium storing instructions for cell therapy, the instructions configured to be executed by one or more processors of a system comprising a cell therapy suite, wherein the instructions are configured such that execution of the instructions causes the system to: receive information comprising a library of cell therapy protocols for a plurality of combinations of patient indications and genes of interest (GOI); receive a request for treating a patient with cell therapy based on a combination of the patient's indication and GOI; and in response to receiving the request for treating a patient with cell therapy, perform a step of a respective cell therapy patient protocol from the library of cell therapy protocols that corresponds to the combination of the patient's indication and GOI.

In some embodiments, a system of cell therapy treatment includes one or more processors configured to: receive a cell therapy protocol for a combination of a patient's indication and genes of interest (GOI); in response to receiving the cell therapy protocol, displaying instructions how to perform a first step of the cell therapy protocol; determine if the step of the cell therapy protocol is complete; and in response to determining if the step of the cell therapy protocol is complete, displaying instructions how to perform a second step of the cell therapy protocol. In some embodiments, the system includes a heads-up-display (HUD), wherein the instructions how to perform the first step of the cell therapy protocol are displayed on the HUD. In some embodiments, the HUD is an augmented reality HUD, wherein the instructions how to perform the first step of the cell therapy protocol are overlayed onto a component of a cell therapy suite. In some embodiments, the instructions how to perform the first step of the cell therapy protocol identifies the component of the cell therapy suite to be used in the step of the cell therapy protocol. In some embodiments, the HUD is a virtual reality HUD, wherein the instructions how to perform the first step of the cell therapy protocol comprises a simulated cell therapy suite. In some embodiments, the HUD comprises an image capture device, and in response to receiving the cell therapy protocol, the image capture device captures images of a cell therapy suite. In some embodiments, determining if the first step of the cell therapy protocol is complete comprises receiving a user input indicated the step of the cell therapy protocol is complete. In some embodiments, determining if the first step of the cell therapy protocol is complete comprises determining if a user performs a gesture corresponding to the first step of the cell therapy protocol. In some embodiments, in response to receiving the cell therapy protocol, providing auditory instructions how to perform the first step of the cell therapy protocol. In some embodiments, in response to determining if the step of the cell therapy protocol is complete, providing auditory or haptic feedback.

In some embodiments, a method for cell therapy performed at a system that includes one or more processors includes: receiving information comprising a cell therapy protocol for a combination of a patient's indication and genes of interest (GOI); in response to receiving the cell therapy protocol, displaying instructions how to perform a first step of the cell therapy protocol; determining if the step of the cell therapy protocol is complete; and in response to determining if the step of the cell therapy protocol is complete, displaying instructions how to perform a second step of the cell therapy protocol.

In some embodiments, a non-transitory computer readable medium storing instructions for cell therapy includes instructions configured to be executed by one or more processors, wherein the instructions are configured such that execution of the instructions causes the system to: receive a cell therapy protocol for a combination of a patient's indication and genes of interest (GOI); and in response to receiving the cell therapy protocol, display instructions how to perform a first step of the cell therapy protocol; and determine if the step of the cell therapy protocol is complete; and in response to determining if the step of the cell therapy protocol is complete, display instructions how to perform a second step of the cell therapy protocol.

In some embodiments, a system for cell therapy treatment includes a cell therapy suite; and one or more processors configured to: receive a cell therapy protocol for a combination of a patient's indication and genes of interest (GOI); in response to receiving the cell therapy protocol, capture images of the cell therapy suite and/or patient; send the images of the cell therapy suite and/or patient to a location remote from the cell therapy suite; and in response to sending the images of the cell therapy suite, receive instructions how to perform a step of the cell therapy protocol. In some embodiments, the instructions how to perform the step of the cell therapy protocol are from the location outside the cell therapy suite. In some embodiments, the instructions how to perform the step of the cell therapy protocol are from a remote operator viewing the images of the cell therapy suite and/or patient at the location outside the cell therapy suite. In some embodiments, the images of the cell therapy suite comprise an image of a component identifier of a component of the cell therapy suite, wherein the instructions how to perform a step of the cell therapy protocol comprises instructions how to operate the component of the cell therapy suite. In some embodiments, the component identifier is a QR code or a barcode corresponding to the component of the cell therapy suite. In some embodiments, the images of the cell therapy suite and/or patient are video images of the cell therapy suite. In some embodiments, the one or more processors are configured to display the instructions how to perform a step of the cell therapy protocol in response to receiving instructions how to perform the step of the cell therapy protocol. In some embodiments, the system includes a heads-up-display (HUD), wherein the instructions how to perform the first step of the cell therapy protocol are displayed on the HUD. In some embodiments, the HUD is an augmented reality HUD, wherein the instructions how to perform the step of the cell therapy protocol are overlayed onto a component of a cell therapy suite.

In some embodiments, a method for cell therapy performed at a system that includes a cell therapy suite and one or more processors includes receiving a cell therapy protocol for a combination of a patient's indication and genes of interest (GOI); in response to receiving the cell therapy protocol, capturing images of the cell therapy suite and/or patient; sending the images of the cell therapy suite and/or patient to a location remote from the cell therapy suite; and in response to sending the images of the cell therapy suite, receiving instructions how to perform a step of the cell therapy protocol.

A non-transitory computer-readable medium storing instructions for cell therapy, the instructions are configured to be executed by one or more processors of a system comprising a cell therapy suite, wherein the instructions are configured such that execution of the instructions causes the system to: receive a cell therapy protocol for a combination of a patient's indication and genes of interest (GOI); in response to receiving the cell therapy protocol, capture images of the cell therapy suite and/or patient; send the images of the cell therapy suite and/or patient to a location remote from the cell therapy suite; and in response to sending the images of the cell therapy suite, receive instructions how to perform a step of the cell therapy protocol.

In some embodiments, any one or more of the characteristics of any one or more of the systems, methods, suites, and/or computer-readable storage mediums recited above may be combined, in whole or in part, with one another and/or with any other features or characteristics described elsewhere herein.

### BRIEF DESCRIPTION OF THE FIGURES

Exemplary embodiments are described with reference to the accompanying figures, in which:

FIG. 1 depicts a schematic diagram of a system for cell therapy guidance, in accordance with some embodiments.

FIG. 2 depicts a flowchart depicting a method for providing cell therapy guidance, in accordance with some embodiments.

FIG. 3 depicts a second flowchart depicting a method for providing cell therapy guidance, in accordance with some embodiments.

FIG. 4 illustrates an example of an augmented reality or virtual reality HUD in accordance with some embodiments.

FIG. 5 depicts a computer, in accordance with some embodiments.

### DETAILED DESCRIPTION

Reference will now be made in detail to implementations and embodiments of various aspects and variations of systems and methods described herein. Although several exemplary variations of the systems and methods are described herein, other variations of the systems and methods may include aspects of the systems and methods described herein combined in any suitable manner having combinations of all or some of the aspects described.

Disclosed herein are systems, methods, computer readable mediums, and techniques that may address one or more of the needs discussed above. Described herein are exemplary embodiments of systems for providing cell therapy in a suite, which may address the problems and shortcomings of known systems and methods described above.

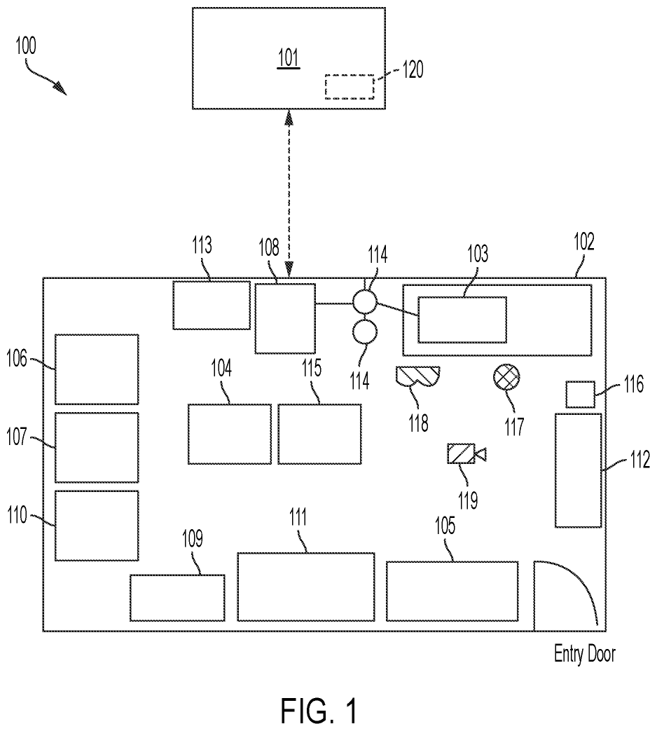

FIG. 1 shows system 100 for communicating information (e.g., data, commands, etc.) between a cell therapy provider system 101 and a cell therapy suite 102, in accordance with some embodiments. As shown, system 100 may include a cell therapy suite where cell therapy treatments can be performed on patients or a plurality of cell therapy suites. In some embodiments, a system may not include a cell therapy suite. The suite can be a suite used in a hospital or other medical treatment facility to treat patients using cell therapy. In some embodiments, the suite can be a standalone suite to treat patients using cell therapy that is separate from a hospital or other medical treatment facility. For example, the suite can be a modular standalone mini-clinic for cell therapy. In some embodiments, the cell therapy suite can be in any facility as a standalone suite for providing cell therapy services. For example, the cell therapy suite may be installed in a pharmacy or may be constructed as a mobile unit that can visit hospitals or other medical treatment facilities as the need for a cell therapy service arises. These suites can be a clean room that satisfies ISO 5, ISO 7, and/or ISO 8.

Some or each of the components in FIG. 1 may be communicatively coupled with one another such that they may send and receive electronic information via network communication amongst one another. As shown in the example of FIG. 1, cell therapy provider system 101 may be communicatively coupled to cell therapy suite 102. Information may be transferred from the cell therapy provider system 101 to the cell therapy suite 102 and vice versa in order to provide the components (e.g., cell-processing device, computer, display, apheresis device, sequencer, RNA production unit, etc.) located in or outside the suite with the most up-to-date information (i.e., instructions, protocols, settings, data, etc.) for performing or training cell therapy on a patient in or outside the suite for a given session/day. In some embodiments, the cell therapy provider system can also send information (e.g., maintenance/update pings, commands, etc.) to any and all components (refrigerator(s), freezer(s), incubator(s), apheresis device, etc.) in or outside the suite. In some embodiments, a cell therapy provider can push updates to the electronic information sent from the cell therapy provider system to the component(s) in or outside the cell therapy suite. This may be done either automatically or manually by personnel or remote operator 120 (e.g., a supervisor of an operator in or outside the suite or a supervising system) of the cell therapy provider. In some embodiments, the cell therapy provider system can include a remote operator 120 (e.g., a supervisor of an operator in or outside the suite). In some embodiments, the remote operator can be at a location outside of the suite or a location remote from the suite and/or operator. In some embodiments, the remote operator can be a part of a separate system 120 that is communicatively coupled to cell therapy provider system and/or cell therapy suite 102. Information may be transferred from the remote operator 120 to the cell therapy provider system 101 and/or to the cell therapy suite 102 and vice versa in order to provide the components (e.g., cell-processing device, computer, display, apheresis device, sequencer, RNA production unit, etc.) located in or outside the suite with the most up-to-date information (i.e., instructions, protocols, settings, data, etc.) for performing or training cell therapy in or outside the suite for a given session/day. In some embodiments, the remote operator can also send information (e.g., maintenance/update pings, commands, etc.) to any and all components (refrigerator(s), freezer(s), incubator(s), apheresis device, etc.) either through cell therapy provider system and/or through remote operator himself/herself in or outside the suite. In some embodiments, a remote operator can push updates to the electronic information sent either through cell therapy provider system and/or through remote operator himself/herself to the component(s) in or outside the cell therapy suite.

In some embodiments, some or each of the components in FIG. 1 may be communicatively coupled with one another such that they may send and receive electronic information via network communication amongst one another without needing a cell therapy suite. For example, cell therapy provider system 101 (and/or remote operator) may be communicatively coupled to a computer(s) or other components outside of the cell therapy suite. Information may be transferred from the cell therapy provider system 101 (and/or remote operator) to such a computer(s) or other components and vice versa in order to provide the components located outside the suite with the most up-to-date information (i.e., instructions, protocols, settings, data, etc.) for performing cell therapy on a patient. In some embodiments, the cell therapy provider system (and/or remote operator) can also send information (e.g., maintenance/update pings, commands, etc.) to any and all components outside the suite. In some embodiments, a cell therapy provider (and/or remote operator) can push updates to the electronic information sent from the cell therapy provider system (and/or remote operator) to the component(s) outside the cell therapy suite. This may be done either automatically or manually by personnel of the cell therapy provider.

In some embodiments, the cell therapy suite can include at least one of a plurality of other components such as a cell-processing device 103, an apheresis device 104, a computer 105, refrigerator(s) 106, freezer(s) 107, an incubator 108, a sequencer 109, an RNA production unit 110, a biosafety hood 111, display 118, imaging device 119, and a storage rack 112. In some embodiments, any and all of these components may be outside of a suite. Any and all of the components in the suite can be communicatively coupled with one another such that they may send and receive electronic information via network communication amongst one another. In some embodiments, only some of the components in the suite can be communicatively coupled with one another such that they may send and receive electronic information via network communication amongst one another. For example, the following information can be communicated between the various components in or outside the suite: relevant data specific to the patient; suite environment data; technician training information and materials; timing data; and/or “which step in the procedure” data is shared between the components, or is distributed by the computer within the suite to synchronize the therapy's components for the patient. In addition, since the therapies can also be done in a staggered manner (one patient's cells are in the incubator while treating another with another device such as the cell-processing device or apheresis device), the tracking and organization of procedure and patient data can also be communicated between the various components in the suite. Any and all of this information can be in a library of cell therapy protocols. These cell therapy protocols can be for a plurality of combinations of patient indications and genes of interest (GOI). In some embodiments, the library can include at least one cell therapy protocol. In some embodiments, a cell therapy protocol can be for at least one combination of a patient indication(s) and a GOI(s). In some embodiments, a GOI can correspond to a cell therapy protocol for one or more patient indications. In some embodiments, a cell therapy protocol for a given patient indication can correspond to one or more GOIs. In some embodiments, any and all of the components in or outside the suite can have a display such as a graphical user interface for displaying any information received by the component or sent from the component. In some embodiments, an operator in or outside the suite can interact with any of these displays such as by touch if it's a touch screen.

In some embodiments, any and all of the components can be outside of a suite and can be communicatively coupled with one another such that they may send and receive electronic information via network communication amongst one another. In some embodiments, only some of the components outside the suite can be communicatively coupled with one another such that they may send and receive electronic information via network communication amongst one another. For example, the following information can be communicated between the various components outside the suite: relevant data specific to the patient; suite environment data; technician training information or materials; timing data; and/or “which step in the procedure” data is shared between the components, or is distributed by the computer within the suite to synchronize the therapy's components for the patient. In addition, since the therapies can also be done in a staggered manner (one patient's cells are in the incubator while treating another with another device such as the cell-processing device or apheresis device), the tracking and organization of procedure and patient data can also be communicated between the various components outside the suite. Any and all of this information can be in a library of cell therapy protocols. These cell therapy protocols can be for a plurality of combinations of patient indications and genes of interest (GOI). In some embodiments, any and all of the components outside the suite can have a display such as a graphical user interface for displaying any information received by the component or sent from the component. In some embodiments, an operator or technician outside the suite can interact with any of these displays such as by touch if it's a touch screen.

In some embodiments, any and all of the components in or outside the suite can be communicatively coupled with cell therapy provider system 101 (and/or remote operator) such that they may send and receive electronic information via network communication amongst one another. In some embodiments, only some of the components in or outside the suite are communicatively coupled with cell therapy provider system 101 (and/or remote operator) such that they may send and receive electronic information via network communication amongst one another. For example, a computer in or outside the suite may be communicatively coupled with cell therapy provider system (and/or remote operator), but the computer may be communicatively coupled to other components in or outside the suite such as the cell processing device such that only the computer communicates with the cell therapy provider system (and/or remote operator), but the computer can communicate with the other components in or outside the suite. As such, the other components in or outside the suite may be able to communicate with the cell therapy provider system (and/or remote operator) through a computer. Any and all of the components in or outside the cell therapy suite may include one or more computer processors configured to send and receive electronic information.

In some embodiments, any and all of the components in or outside the suite can transfer the following information to the cell therapy provider system (and/or remote operator): information to be used for learning and analysis (e.g., machine learning or artificial intelligence); information for assisting and/or guiding an operator or technician (e.g., real-time assistance); information to be used for operator or technician training and/or monitoring; offloaded data processing or live-streamed data or programs for operating the suite (including “where in the procedure”, any errors, live data or data sent from the in-suite computer about the patient); maintenance or update pings or commands for network-connected devices such as refrigerators, incubators, apheresis machine, patient data based on appointment time/verification; augmented reality training programs/execution programs; verification keys for subscription; network timing protocol information; information on shipping, supplies, and timing based on usage in the suite.

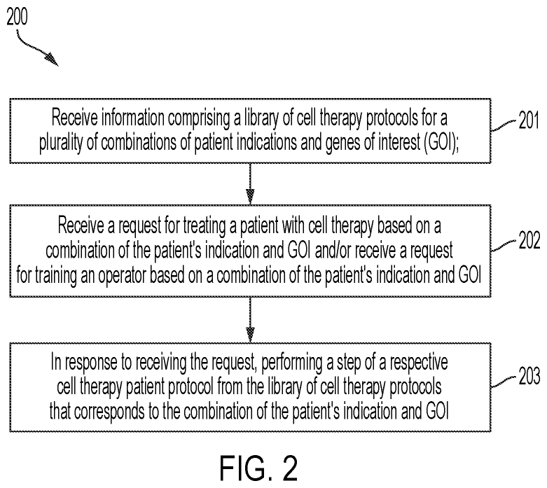

FIG. 2 depicts a flowchart representing an exemplary method 200 for providing cell therapy guidance, in accordance with some embodiments. As described in detail below, method 200 may enable a system (such as system 100) to receive information comprising a library of cell therapy protocols for a plurality of combinations of patient indications and genes of interest (GOI), peptides, or small molecules; receive a request for treating a patient with cell therapy based on a combination of the patient's indication and GOI, peptide, or small molecule and/or receive a request for training an operator based on a combination of the patient's indication and GOI, peptide, or small molecule; and in response to receiving the request for treating a patient with cell therapy, performing a step of a respective cell therapy patient protocol from the library of cell therapy protocols that corresponds to the combination of the patient's indication and GOI, peptide, or small molecule.

In some embodiments, method 200 may be carried out, in whole or in part, by one or more of the components of a system, such as system 100 described in FIG. 1 and the corresponding description. In some embodiments, any one or more of the aspects of method 200 may be combined, in whole or in part, with any one or more of the components of FIG. 1, and/or with any one or more of the systems, methods, devices, and/or techniques described elsewhere herein.

At block 201, in some embodiments, the system may receive information comprising a library of cell therapy protocols (or portion thereof) for a plurality of combinations of patient indications and genes of interest (GOI), peptides, or small molecules. In some embodiments, the library of cell therapy protocols can include at least one combination of patient indication(s) and GOI, peptides, or small molecules. In some embodiments, the library of cell therapy protocols can be a single cell therapy protocol for a specific combination of a patient's indication and GOI, peptides, or small molecules. In some embodiments, the library of cell therapy protocols can include at a least a portion or step of a cell therapy protocol or an update to at least a portion/step of a cell therapy protocol. For example, when the library of cell protocols is updated, one or more cell protocols (or steps thereof) may be sent/received to update the library. In other words, the entire library does not need to be sent/received at all times. Instead, only a portion thereof can be sent/received. In some embodiments, a cell therapy protocol may be a training cell therapy protocol used to teach an operator how to perform cell therapy on a patient. As such, the cell therapy protocol may be a training version of an actual cell therapy protocol. For example, the cell therapy protocol may not include all steps of an actual cell therapy patient protocol as there may not be a patient when an operator is training to use a cell therapy suite.

In some embodiments, the cell therapy provider system can send information to any and all individual components in or outside the suite related to the following which are discussed in more detail herein: protocols for the cell-processing device; maintenance or update pings or commands for network-connected devices such as refrigerators, incubators, and/or apheresis machine; patient data based on appointment time/verification; training programs (e.g., Augmented or Virtual Reality training programs/execution programs); real-time assistance/guidance including real-time record keeping (e.g., video recording, scanning of barcodes (QR), digitized checklists); verification keys for subscription; network timing protocol information; plasmid DNA sequence and RNA sequence for production; information for tuning power usage for a smart UPS; and/or information on shipping, supplies, and timing based on usage in the suite, which can be tracked by weight change in the storage rack, scanning of barcodes by either the cell-processing device or using the built-in computer in the suite, or by a camera or other sensor detecting the usage of the reagent or material. Any and all of this information can be included in the library of cell therapy protocols. Any and all of this information can be stored entirely in an in-suite or out-of-suite computer, in the memory of the individual components in or outside the suite, or it can stay in the cell therapy provider system (and/or remote operator).

In some embodiments, cell therapy provider system 101 and remote operator 120 may be any device or system comprising one or more computer processors configured to send and receive electronic information. In some embodiments, cell therapy provider system 101 and remote operator 120 may be provided, in whole or in part, as all or part of a desktop computing device, laptop, tablet, mobile electronic device, computing module, processor, server, cloud computing system, distributed computing system, or the like. In some embodiments, cell therapy provider system 101 and remote operator 120 may be provided locally with respect to a cell therapy suite 102 (e.g., in the suite), while in some embodiments cell therapy provider system 101 and remote operator 120 may be provided remotely from suite 102 (e.g., outside the suite, at a remote server location, etc.).

In the example of system 100, at least one of the components in or outside suite 102 can be configured to communicate (e.g., by any suitable wired or wireless network communication protocol) with cell therapy provider system 101 (and/or remote operator). In some embodiments, a component in or outside suite 102 can be configured to communicate (e.g., by any suitable wired or wireless network communication protocol) with any or all other components in or outside suite 102. In some embodiments, non-computer 105 components in or outside suite 102 may be configured to only communicate with the computer 105. In some embodiments, only computer 103 (which may be outside the suite) may be configured to communicate with cell therapy provider system 101 (and/or remote operator). In some embodiments, components in or outside suite 102 may be configured to send and/or receive information directly to and from one or more other components in or outside suite 102 and/or to cell therapy provider system 101 (and/or remote operator). In some embodiments, any or all components in or outside suite 102 can be communicatively coupled to receive or send information to any or all components in or outside another suite 102. Communicatively coupled can mean that the components are capable of communicating by any wired or wireless electronic communication medium, including by any suitable network communication protocol.

As explained above, the suite may include an apheresis device 104 for extracting human cells (e.g., blood cells). The apheresis device can be a conventional apheresis device such as the Terumo Spectra Optia. In some embodiments, the apheresis device can be a computer controlled and/or automated apheresis device for extracting patient's cells. For example, the apheresis device can draw patient blood, separate blood components to an extent, and/or fill these components into a blood bag or similar bag for use in the next component in the suite for cell therapy. As such, a patient can go to a cell therapy suite disclosed herein for collection of blood cells using the apheresis device. In some embodiments, apheresis of the patient may be performed in a different suite or remote location outside the suite and brought into the suite for cell processing.

As explained, the suite or a location outside the suite can include one or more processors configured to send and receive electronic information such as the library of cell therapy protocols. In some embodiments, the electronic information including the library of cell therapy protocols can include maintenance/update pings or commands for the apheresis device. For example, the apheresis device can receive apheresis procedure information for which procedure the patient is undergoing and/or the settings to perform apheresis for the specific patient. In some embodiments, the cell therapy provider system (and/or remote operator) is configured to send the electronic information (e.g., the library of cell therapy protocols) to the apheresis device itself in the suite. In some embodiments, the cell therapy provider system (and/or remote operator) can send the apheresis procedure information for the apheresis device to the computer in or outside the suite. The computer can then relay this information to the apheresis device or the computer can store this information and thereby control the cell-processing device. The cell therapy provider system (and/or remote operator) can update and/or maintain this information for the apheresis device. For example, using the network connection, a cell therapy provider can push updates to the apheresis settings from the cell therapy provider system (and/or remote operator) to the computer (the computer can then send the information to the apheresis device) and/or apheresis device in or outside the cell therapy suite. This may be done either automatically or manually by personnel of the cell therapy provider. Personnel of a cell therapy system can include a physician, nurse, technician, etc., or supervisor thereof or even an individual who knows how to perform a cell therapy protocol, is trained on performing a cell therapy protocol, or is even employed by the cell therapy provider. In some embodiments, the personnel of a cell therapy system can include an individual who is not affiliated with the cell therapy provider. In some embodiments, the remote operator can be considered to be personnel of the cell therapy provider.

At block 202, in some embodiments, the system may receive a request for treating a patient with cell therapy based on a combination of the patient's indication and GOI, peptide, or small molecule and/or the system may receive a request for training an operator of a cell therapy system. At block 203, in some embodiments, the system may in response to receiving the request for treating a patient with cell therapy and/or training an operator of a cell therapy system, perform a step of a respective cell therapy patient protocol from the library of cell therapy protocols that corresponds to the combination of the patient's indication and GOI, peptide, or small molecule. For example, in response to receiving the apheresis procedure information and/or in response to receiving a request from an operator, the apheresis device can perform an apheresis procedure (from the library of cell therapy protocols) such as extracting a patient's blood cells.

After apheresis, the operator can load the patient's blood cells and a vector (such as adeno-associated virus, DNA plasmid, RNA, self-replicating or self-amplifying RNA, or c-srRNA) expressing a GOI, a peptide, or small molecule for treating an indication onto the cell-processing device in the suite. The cell-processing device can be a computer controlled and/or automated cell-processing device for processing patient's cells (e.g., human cells). In some embodiments, the cell-processing device can be a CliniMACS Prodigy® device. Examples and descriptions of such cell-processing devices can be found in U.S. Pat. Nos. 10,828,333; 10,350,245; 10,705,091; 10,705,090; 10,620,212; 10,131,876; 10,119,970; 10,072,245; 9,714,945; 9,625,463; and 9,493,789, which are all hereby incorporated by reference in their entirety.

In some embodiments, the electronic information and/or the library of cell therapy protocols can include at least one or a plurality of operating conditions for the cell-processing device for a plurality of combinations of patient indications and genes of interest (GOI), peptides, or small molecules. This plurality of operating conditions can be a library of operating conditions or protocols for the cell-processing device based on specific indications and GOIs, peptides, or small molecules. As stated above, a patient indication can correspond to one or more GOIs, peptides, or small molecules or vice versa for a given cell therapy protocol. For example, a cell therapy protocol for a patient indication can require utilizing more than one GOI(s), peptide(s), or small molecule(s). In some embodiments, the cell therapy provider system (and/or remote operator) is configured to send electronic information (i.e., the library of cell therapy protocols including the plurality of operating conditions for the cell-processing device) to the suite or any component(s) of the system inside or outside of the suite. The cell therapy provider system (and/or remote operator) can update and/or maintain this library of cell therapy protocols (e.g., operating conditions for the cell-processing device). For example, using the network connection, a cell therapy provider can push updates to the library of cell therapy protocols (e.g., operating conditions from the cell therapy provider system) to the computer and/or cell-processing device in or outside the cell therapy suite. This may be done either automatically or manually by personnel of the cell therapy provider. In some embodiments, the updates may be for a modification of the operating condition for an existing combination of a GOI, peptide, or small molecule and an indication or for addition of an operating condition for a new combination of a GOI, peptide, or small molecule and an indication.

In some embodiments, when the cell therapy suite is installed, the library of cell therapy protocols (e.g., plurality of operating conditions for the cell-processing device) can also be installed in the cell-processing device and/or computer which can contain all the known operating conditions for cell therapies that have been developed or approved at the time of installation. However, as new cell therapies are developed and approved or updates to existing cell therapies, the operating conditions for the cell-processing device when treating such new cell therapies can be created and added to the existing library of operating conditions. In some embodiments, the cell therapy provider system (and/or remote operator) can send these new operating conditions for the cell-processing device. Furthermore, when an operating condition for an existing combination of a GOI, peptide, or small molecule and an indication has been modified to improve the corresponding cell therapy, the existing operating condition for such combination of GOI, peptide, or small molecule and indication can be replaced with the operating condition for the improved therapy. In some embodiments, the cell therapy provider system (and/or remote operator) can send these new operating conditions for the cell-processing device. In some embodiments, updating the library can be made by any conventional method. For example, a technician can update the library of cell therapy protocols in the computer and/or cell-processing device in or outside the suite or remotely from another computer (i.e., remote operator and/or the cell therapy provider system) connected to the suite by loading updates to the library manually and pushing them to the suite. In some embodiments, the computer and/or cell-cell processing device can be connected via network to a server computer (i.e., the cell therapy provider system and/or remote operator) remotely located away from where the suite is installed. Under this configuration, the server computer can automatically or manually push updates to the library of cell therapy protocols (e.g., library of operating conditions for the cell-processing device). In some embodiments, the server computer can contain the library of cell therapy protocols and the operator can access the library in the server computer via network connection. In some embodiments, the operator may need a secure log in or verification code to access the library in the server computer. Under this configuration, the server computer itself can maintain the library of cell therapy protocols (e.g., library of operating conditions) from which the operator chooses the operating condition depending on the combination of the patient's indication and GOI, peptide, or small molecule.

In some embodiments, the cell-processing device can include the library of operating conditions or protocols for the cell-processing device. In some embodiments, the cell therapy provider system (and/or remote operator) can send the plurality of operating conditions for the cell-processing device to the cell-processing device itself. In some embodiments, a computer such as computer 105 that is communicatively coupled to cell-processing device can include the library of operating conditions or protocols for the cell-processing device. In some embodiments, the cell therapy provider system (and/or remote operator) can send the library of cell therapy protocols which can include the plurality of operating conditions for the cell-processing device to the computer in or outside the suite. The computer can then relay this library of operating conditions to the cell-processor device or the computer can store the library of operating conditions and thereby control the cell-processing device. These operating conditions or protocols for the cell-processing device can be designed for a specific indication to be treated by cell therapy and for delivering a gene of interest (GOI), peptide, or small molecule for such treatment. In other words, the operating conditions or protocols for the cell processing device can correspond to a specific combination of the indication and the GOI, peptide, or small molecule. Examples of indications include, but are not limited to, bone marrow diseases, such as those described in U.S. Pat. No. 10,335,456 (“Methods of Using ZSCAN4 for Rejuvenating Human Cells,” by Minoru Ko) and cancers that can be treated in the methods described in U.S. Pat. No. 11,421,248 (“Temperature-based Transient Delivery of Nucleic Acids and Proteins to Cells and Tissue” by Minoru Ko), which are both hereby incorporated by reference in their entirety. One example of a GOI is ZSCAN4, which can be used to treat several indications, such as telomere shortening, karyotype abnormalities and chromosome abnormalities. Accordingly, the operating conditions of the cell-processing device for such indications can be different and optimized for each, even though the same GOI may be used. Other examples of GOIs include, but are not limited to, those for genome editing, CAR T cell therapy, and PDI therapy.

As such, the library of cell therapy protocols can include operating conditions for the cell-processing device designed for a respective combination of a GOI, peptide, or small molecule and indication. An operator (e.g., a physician, nurse, technician, etc.) of the cell-processing device can choose (such as selecting from a touchscreen display or other display) an operating condition from the library at the computer, the cell-processing device, or both depending on where the library is located. In other words, one or more processors (e.g., cell-processing device and/or computer) in or outside the suite can receive a request for treating a patient with cell therapy based on a combination of the patient's indication and GOI, peptide, or small molecule. This request can be a request for an operating condition of the cell-processing device based on a combination of a patient's indication and a GOI, peptide, or small molecule. The request can be input from an operator in the suite. For example, the operator can select the operating condition designed for the specific GOI, peptide, or small molecule and the patient's indication from the library of operating conditions.

In response to receiving the request, the cell processing device can process the cells (e.g., blood cells) of a patient with the GOI, peptide, or small molecule according to the requested operating condition. In some embodiments, in response to receiving the request for the operating condition, the cell-processing device can process cells of the patient with a vector (such as adeno-associated virus, DNA plasmid, RNA, self-replicating or self-amplifying RNA, or c-srRNA) carrying the GOI, peptide, or small molecule in the cell-processing device according to a respective operating condition that corresponds to the combination of the patient's indication and GOI, peptide, or small molecule. For example, the cell-processing device's operating conditions can transfer fluids between different bags for treating/processing cells using a pump or other system, employ centrifugation for cell separation, control temperature of (e.g., heat and/or cool) the centrifugation chamber or other reservoir holding the cells or other reagents/fluids (e.g., cell culture medium, cell separation beads, etc.), isolate cells such as T cells, and/or fill the final product. In some embodiments, the cell-processing device can perform any and all of these function in a closed system.

The delivery of GOIs may be performed using a vector (such as adeno-associated virus, DNA plasmid, RNA, self-replicating or self-amplifying RNA, or c-srRNA). In some embodiments, RNA can be used, specifically a temperature-controllable self-replicating RNA (c-srRNA), examples of which are disclosed in U.S. Pat. No. 11,421,248, which is hereby incorporated by reference in its entirety. The unique feature of the c-srRNA is that the replication can occur at certain temperature range, i.e., permissive temperatures. Accordingly, the GOIs, peptides, or small molecules can be activated (i.e., generated) at the permissive temperature and deactivated (i.e., not generated) at a non-permissive temperature that is outside the permissive temperature range. The activation and the deactivation of the GOIs, peptides, or small molecules may both occur in the cell-processing device. Alternatively, the deactivation may occur by infusing the processed cells back to the patient because the body temperature of a patent is outside the permissive temperature range. The activation and the deactivation may be part of the operation condition or may be controlled independently of the operating condition using the computer and/or the cell-processing device itself. The advantages of using c-srRNA over traditional RNA for use in the cell therapy provided in the suite are as follows. Use of c-sRNA is capable of supporting even larger genes, it can load any therapeutic gene, or even multiple genes, for transfection to the treated cells. In addition, it has a longer and stronger expression than traditional RNA, but can be controllable, unlike self-replicating/self-amplifying RNA. Furthermore, it is non-viral, and can be used in a non-BSL environment such as a minimal GMP suite as opposite to a centralized cell-processing center. Its temperature-controllable feature makes it suitable for precise control during culture on an automated cell-processing device, as opposed to other control technologies that may require another molecule or trigger for their on-off switches. These features make c-srRNA flexibility applicable to almost the totality of indications that can be addressed by cell therapy.

As explained above, the cell therapy suite may include a sequencer 109 for performing sequencing on a specimen that is collected from a cell therapy patient. The sequencer can be a conventional sequencer such as the Illumina NextSeq 550 or Illumina iSeq 100. In some embodiments, the sequencer can be a computer controlled and/or automated sequencer that can be used to identify a sequence of a GOI or peptide for the treatment of an indication. In some embodiments, the patient can come to the cell therapy suite prior to apheresis for identifying a specific GOI or peptide for the cell therapy treatment. In some embodiments, for the case of treating a cancer patient, the sequencing may be performed to identify neoantigens of the patient. The results of sequencing can be stored in the sequencer and/or the computer in or outside the suite. In some embodiments, the results of sequencing can be sent (by either the sequencer itself or the computer) to the cell therapy provider system for storage. When multiple cell therapy suites are installed in a given region, only one or not all of the suites may have a sequencer. Instead, the suites without the sequencer can receive sequencing results over any network communication. In this scenario, a patient can visit one suite for sequencing and another for other treatments/processes such as apheresis and/or reinfusion of the processed blood cells.

In some embodiments, the suite, sequencer, and/or computer can include one or more processors configured to receive/send electronic information (e.g., library of cell therapy protocols). In some embodiments, the electronic information and/or library of cell therapy protocols can include maintenance/update pings or commands for the sequencer. For example, the sequencer can receive a library of cell therapy protocols that can include sequence procedure information for the settings to sequence a specimen for a specific patient. In some embodiments, the cell therapy provider system (and/or remote operator) is configured to send the electronic information to the sequencer itself in or outside the suite. In some embodiments, the cell therapy provider system (and/or remote operator) can send a library of cell therapy protocols that includes sequence information for the sequencer to the computer in or outside the suite. The computer can then relay this information to the sequencer or the computer can store this information and thereby control the sequencer. The cell therapy provider system (and/or remote operator) can update and/or maintain this information for the sequencer. For example, using the network connection, a cell therapy provider can push updates to the sequencer settings from the cell therapy provider system (and/or remote operator) to the computer (the computer can then send the information to the apheresis device) and/or sequencer in the cell therapy suite. This may be done either automatically or manually by personnel of the cell therapy provider. In response to receiving the sequence procedure information and/or in response to receiving a request from an operator, the sequencer can perform sequencing to identify a GOI or peptide for the treatment of an indication. As such, a patient can visit the cell therapy suite for biopsy for the identification of the GOI or peptide.

As explained above, the cell therapy suite may include a RNA production unit 110 for producing RNA carrying the GOI, peptide, or small molecule for treatment of an indication. In some embodiments, the RNA production unit can actually be a vector producing unit that is configured to produce a vector (such as adeno-associated virus, DNA plasmid, RNA, self-replicating or self-amplifying RNA, or c-srRNA) expressing a GOI, a peptide, or small molecule for treating an indication onto the cell-processing device in the suite. The RNA production unit can be a self-contained, standalone machine which, given a supply of reagents and sequence and protocol data, can perform all steps necessary to produce RNA in a filled format at either research use or GMP-grade levels. In some embodiments, the RNA production unit can be a computer controlled and/or automated RNA production unit. In some embodiments, the RNA production unit can include one or more processors configured to receive/send electronic information. In some embodiments, the electronic information can include the GOI or peptide identified by the sequencer. In some embodiments, the electronic information can be sent from the sequencer itself, a computer, and/or the cell therapy provider system (and/or remote operator). In response to receiving information about the GOI or peptide identified by the sequencer, the RNA production unit can load the identified GOI or peptide onto a vector or vehicle. In some embodiments, the RNA production unit can load the identified GOI onto an vector in response to operator input. In some embodiments, the RNA production unit can be preconfigured to produce the backbone of a c-srRNA vehicle. The RNA production unit can produce c-srRNA by loading the identified GOI, peptide, or small molecule onto the backbone of the c-srRNA vehicle. In addition, the RNA production unit can include creation of plasmid DNA and capping of the final RNA products.

When the GOI or peptide is identified by a sequencer in a suite, the information about the GOI or peptide can be transmitted locally to the RNA production unit in the same suite. In some embodiments, a suite may not have an RNA production unit. Thus, a cell therapy suite having a sequencer (but not an RNA production unit) can transmit information on the sequence of the GOI or peptide obtained by its own sequencer to an RNA production unit of another cell therapy suite for production of RNA. In some embodiments, the sequencer can send the information to the computer in or outside the cell therapy suite, which sends the information to the cell therapy provider system (and/or remote operator), which then sends the information to a suite containing an RNA production unit.

In some embodiments, the electronic information and/or the library of cell therapy protocols can include maintenance/update pings or commands for the RNA production unit. For example, the RNA production unit can receive a library of cell therapy protocols that includes RNA production procedure (or production procedure of other vectors disclosed herein) information for the settings to produce RNA for a specific patient. In some embodiments, the cell therapy provider system is configured to send the electronic information to the RNA production unit itself in the suite. In some embodiments, the cell therapy provider system can send the RNA information for the RNA production unit to the computer in the suite. The computer can then relay this information to the RNA production unit or the computer can store this information and thereby control the RNA production unit. In some embodiments, the cell therapy provider system (and/or remote operator) can update and/or maintain this information for the RNA production unit. For example, using the network connection, a cell therapy provider can push updates to the information including the RNA production settings from the cell therapy provider system (and/or remote operator) to the computer (the computer can then send the information to the RNA production unit) and/or RNA production unit in or outside the cell therapy suite. This may be done either automatically or manually by personnel of the cell therapy provider.

As explained above, the cell therapy suite may include a refrigerator(s) 106 for storing reagents and/or samples used for cell therapy treatment. The refrigerator can be a conventional refrigerator or a plurality of refrigerators such as a refrigerators capable of reaching temperature of 4° C. and −20° C., which can be stacked. In some embodiments, the refrigerator(s) can be a computer controlled and/or automated refrigerator.

In some embodiments, the suite, refrigerator(s), and/or computer can include one or more processors configured to receive electronic information such as a library of cell therapy protocols. In some embodiments, the electronic information and/or the library of cell therapy protocols can include maintenance/update/settings pings or commands for the refrigerator(s). For example, the refrigerator can receive information for the temperature settings to store a sample for a patient. In some embodiments, the cell therapy provider system (and/or remote operator) is configured to send the electronic information to the refrigerator itself in the suite. In some embodiments, the cell therapy provider system (and/or remote operator) can send the information for the refrigerator to the computer in or outside the suite. The computer can then relay this information to the refrigerator or the computer can store this information and thereby control the refrigerator. The cell therapy provider system can update and/or maintain this information for the refrigerator. For example, using the network connection, a cell therapy provider can push updates to the refrigerator settings from the cell therapy provider system (and/or remote operator) to the computer (the computer can then send the information to the refrigerator) and/or refrigerator(s) in the cell therapy suite. This may be done either automatically or manually by personnel of the cell therapy provider. In response to receiving the information and/or in response to receiving a request from an operator, the refrigerator can adjust its settings.

As explained above, the cell therapy suite may include a freezer(s) 107 for storing reagents and/or samples used for cell therapy treatment. The freezer can be a conventional freezer or a plurality of freezers such as a freezer capable of reaching temperature of −80° C. In some embodiments, the freezer(s) can be a computer controlled and/or automated freezer.

In some embodiments, the suite, freezer(s), and/or computer can include one or more processors configured to receive/send electronic information such as the library of cell therapy protocols. In some embodiments, the electronic information and/or library of cell therapy protocols can include maintenance/update/settings pings or commands for the freezer(s). For example, the freezer can receive information for the temperature settings to store a sample for a patient. In some embodiments, the cell therapy provider system (and/or remote operator) is configured to send the electronic information to the freezer itself in the suite. In some embodiments, the cell therapy provider system (and/or remote operator) can send the information for the freezer to the computer in or outside the suite. The computer can then relay this information to the freezer or the computer can store this information and thereby control the freezer. The cell therapy provider system (and/or remote operator) can update and/or maintain this information for the freezer. For example, using the network connection, a cell therapy provider can push updates to the refrigerator settings from the cell therapy provider system (and/or remote operator) to the computer (the computer can then send the information to the freezer) and/or freezer(s) in the cell therapy suite. This may be done either automatically or manually by personnel of the cell therapy provider. In response to receiving the information and/or in response to receiving a request from an operator, the freezer can adjust its settings.

As explained above, the cell therapy suite may include a biosafety hood 111. The biosafety hood may be used during some cell therapy procedures such as to do sterile connected in the biosafety hood. In some embodiments, the biosafety hood can be a miniature biosafety hood. The biosafety hood can be any conventional biosafety hood. In some embodiments, the biosafety hood can be a computer controlled and/or automated biosafety hood.

In some embodiments, the suite, biosafety hood, and/or computer can include one or more processors configured to receive/send electronic information such as the library of cell therapy protocols. In some embodiments, the electronic information and/or library of cell therapy protocols can include maintenance/update/settings pings or commands for the biosafety hood. For example, the biosafety hood can receive information for a certain setting tied to a cell therapy procedure for a patient. In some embodiments, the cell therapy provider system (and/or remote operator) is configured to send the electronic information to the biosafety hood itself in the suite. In some embodiments, the cell therapy provider system (and/or remote operator) can send the information for the biosafety hood to the computer in or outside the suite. The computer can then relay this information to the biosafety hood or the computer can store this information and thereby control the biosafety hood. The cell therapy provider system (and/or remote operator) can update and/or maintain this information for the biosafety hood. For example, using the network connection, a cell therapy provider can push updates to the refrigerator settings from the cell therapy provider system (and/or remote operator) to the computer (the computer can then send the information to the biosafety hood) and/or biosafety hood in the cell therapy suite. This may be done either automatically or manually by personnel of the cell therapy provider. In response to receiving the information and/or in response to receiving a request from an operator, the biosafety hood can adjust its settings.

As explained above, the cell therapy suite may include an incubator 108 for incubating cells for cell therapy treatment. The incubator can be a conventional incubator. In some embodiments, the incubator can be a computer controlled and/or automated incubator. In some embodiments, the incubator can be used for longer-term storage of cells. In addition, the cells can be stored at a set temperature and/or CO₂ percentage environment. For example, in the case of a multi-day/week process such as CAR-T cell therapy, the cells can be incubated in the incubator at about 33° C., instead of incubated in the cell-processing device. In some embodiments, the cells can be incubated in the cell-processing device.

In some embodiments, the suite, incubator, and/or computer can include one or more processors configured to receive/send electronic information such as the library of cell therapy protocols. In some embodiments, the electronic information and/or library of cell therapy protocols can include maintenance/update/settings pings or commands for the incubator. For example, the incubator can receive information for the cell incubation settings for a patient. In some embodiments, the cell therapy provider system (and/or remote operator) is configured to send the electronic information to the incubator itself in the suite. In some embodiments, the cell therapy provider system (and/or remote operator) can send the information for the incubator to the computer in or outside the suite. The computer can then relay this information to the incubator or the computer can store this information and thereby control the incubator. The cell therapy provider system (and/or remote operator) can update and/or maintain this information for the incubator. For example, using the network connection, a cell therapy provider can push updates to the incubator settings from the cell therapy provider system (and/or remote operator) to the computer (the computer can then send the information to the incubator) and/or incubator in the cell therapy suite. This may be done either automatically or manually by personnel of the cell therapy provider. In response to receiving the information and/or in response to receiving a request from an operator, the incubator can adjust its settings.

As explained above, the cell therapy suite may include a storage rack 112 for storing reagents and various other materials for a cell therapy treatment process. The storage rack can be a conventional storage rack. In some embodiments, the storage rack may have sensors (e.g., weight sensors) to track of the amount of the various materials on the storage rack. As such, the storage rack may be configured to keep track of usage of the various materials used in a cell therapy treatment process. In some embodiments, the storage rack can be a computer controlled and/or automated storage rack.

In some embodiments, the suite, storage rack, and/or computer can include one or more processors configured to receive/send electronic information such as the library of cell therapy protocols. In some embodiments, the storage rack can send information on the amount of a material stored on the storage rack to the computer (and then to the cell therapy provider system) and/or cell therapy provider system (and/or remote operator). In some embodiments, the electronic information and/or library of cell therapy protocols can include maintenance/update/settings pings or commands for the storage rack. In some embodiments, the cell therapy provider system (and/or remote operator) is configured to send the electronic information to the storage rack itself in the suite. In some embodiments, the cell therapy provider system (and/or remote operator) can send the information for the storage rack to the computer in or outside the suite. The computer can then relay this information to the storage rack or the computer can store this information and thereby control the storage rack. The cell therapy provider system (and/or remote operator) can update and/or maintain this information for the storage rack. For example, using the network connection, a cell therapy provider can push updates to the storage rack settings from the cell therapy provider system (and/or remote operator) to the computer (the computer can then send the information to the storage rack) and/or storage rack in the cell therapy suite. This may be done either automatically or manually by personnel of the cell therapy provider. In response to receiving the information and/or in response to receiving a request from an operator, the storage rack can adjust its settings and/or send additional information.

In some embodiments, the cell therapy suite may include a power supply 113 for powering the suite and the various components of the suite. The power supply can be any conventional power supply source such as a battery, gas/diesel generator, solar power source, wind power source, or other energy source powered generator. In some embodiments, the power supply can be an uninterruptible power supply. In some embodiments, the power supply can provide stabilized power for proper functioning of all the components in the suite. In some embodiments, the power supply can be a computer controlled and/or automated storage power supply. The suite having its own power supply can allow the suite to be a standalone unit.

In some embodiments, the suite, power supply, and/or computer can include one or more processors configured to receive and/or send electronic information such as the library of cell therapy protocols. In some embodiments, the electronic information and/or library of cell therapy protocols can include maintenance/update/settings pings or commands for the power supply. In some embodiments, the cell therapy provider system (and/or remote operator) is configured to send the electronic information to the power supply itself in the suite. In some embodiments, the cell therapy provider system (and/or remote operator) can send the information for the power supply to the computer in or outside the suite. The computer can then relay this information to the power supply or the computer can store this information and thereby control the power supply. The cell therapy provider system (and/or remote operator) can update and/or maintain this information for the power supply. For example, using the network connection, a cell therapy provider can push updates to the power supply settings such as information for tuning power usage for a smart uninterruptible power supply from the cell therapy provider system (and/or remote operator) to the computer (the computer can then send the information to the power supply) and/or power supply in the cell therapy suite. This may be done either automatically or manually by personnel of the cell therapy provider. In response to receiving the information and/or in response to receiving a request from an operator, the power supply can adjust its settings.

In some embodiments, the cell therapy suite can include gas supply or gas supplies such as CO₂ and/or nitrogen cylinders to be used in cell-processing and/or incubation. In some embodiments, the gas supply or supplies can be outside the suite with feed tubes to the cell-processing device and/or incubator. In some embodiments, the gas can be supplied to the cell-processing device and/or incubator based on the information received from the cell-processing device and/or incubator. In some embodiments, the gas supply or supplies can be a part of the cell-processing device and/or incubator. In some embodiments, the gas supply or supplies can be communicatively coupled to any and all components in or outside the suite and/or the cell therapy provider system to send and/or receive electronic information.

In some embodiments, the cell therapy suite can include a patient chair 115, a trash receptacle 116, and an operator chair 117. The patient chair can be where the patient sits during the cell therapy treatment. The operator chair can be where the operator sits during the cell therapy treatment. The trach receptacle can be where any trash and/or biohazard waste can be stored for later removal from the suite. In some embodiments, the patient chair, trash receptacle, and/or operator chair can be communicatively coupled to any and all components in or outside the suite and/or the cell therapy provider system (and/or remote operator) to send and/or receive electronic information. In some embodiments, the cell therapy suite can include a sterile connection device (not shown) such as the one by Terumo. The sterile connection device can be used to connect tubes together. In some embodiments, the sterile connection device can be used to connect tubes together without the need for a biosafety hood/cabinet. In some embodiments, the sterile connection device can be communicatively coupled to any and all components in or outside the suite and/or the cell therapy provider system to send and/or receive electronic information.

In some embodiments, the cell therapy suite may include one or more displays 118 (e.g, a graphical user interface) for displaying any information received from or sent to the any of the components in or outside the suite. In some embodiments, the one or more displays can be outside or remote from a cell therapy suite. As explained in more detail herein, in some embodiments, the display can be a heads-up-display (HUD). In some embodiments, the at least one display can provide visual cues as to performing steps of a cell therapy protocol. In some embodiments, the at least one display can also provide audio cues as to performing steps of a cell therapy protocol. In some embodiments, the suite, display(s), and/or computer can include one or more processors configured to receive/send electronic information such as the library of cell therapy protocols. In some embodiments, the display can send information on what step of the cell therapy process the operator is on to the computer (and then to the cell therapy provider system) and/or cell therapy provider system (and/or remote operator). In some embodiments, the electronic information and/or library of cell therapy protocols can include maintenance/update/settings pings or commands for the display(s). In some embodiments, the cell therapy provider system (and/or remote operator) is configured to send the electronic information to the display itself. In some embodiments, the cell therapy provider system (and/or remote operator) can send the information for the display(s) to the computer in or outside the suite. The computer can then relay this information to the display(s) or the computer can store this information and thereby control the display(s). The cell therapy provider system (and/or remote operator) can update and/or maintain this information for the display. For example, using the network connection, a cell therapy provider can push updates to the display settings from the cell therapy provider system (and/or remote operator) to the computer (the computer can then send the information to the display) and/or display in or outside the cell therapy suite. This may be done either automatically or manually by personnel of the cell therapy provider. In response to receiving the information and/or in response to receiving a request from an operator, the display can adjust its settings, display certain information, and/or send additional information.

In some embodiments, the information comprising the library of cell therapy protocols comprises instructions how to perform at least one step or more than one step of the respective cell therapy protocol from the library of cell therapy protocols that corresponds to the combination of the patient's indication and GOI. In some embodiments, in response to receiving the request for treating a patient with cell therapy, the system can display the instruction how to perform the at least one step of the respective cell therapy protocol from the library of cell therapy protocols that corresponds to the combination of the patient's indication and GOI.

In some embodiments, the cell therapy suite may include one or more image capturing devices 119 (e.g, a camera) for capturing images of anything in the cell therapy suite (e.g., components of the cell therapy suite, the patient(s), materials used by the components of the cell therapy suite). In some embodiments, the images can be a video stream. In some embodiments, in response to receiving the request for treating a patient with cell therapy, the imaging device(s) can capture images of the cell therapy suite. In some embodiments, the imaging device can be a component of a heads-up-display (e.g., a front facing camera of a HUD). In some embodiments, the one or more image capturing devices can be outside or remote from a cell therapy suite. In some embodiments, the suite, image capturing device(s), and/or computer can include one or more processors configured to receive/send electronic information such as the library of cell therapy protocols. In some embodiments, the at least one image capturing device can capture images (and send) on what step of the cell therapy process the operator is on to the computer (and then to the cell therapy provider system) and/or cell therapy provider system (and/or remote operator). In some embodiments, the electronic information and/or library of cell therapy protocols can include maintenance/update/settings pings or commands for the at least one image capturing device. In some embodiments, the cell therapy provider system (and/or remote operator) is configured to send the electronic information to the at least one image capturing device itself. In some embodiments, the cell therapy provider system (and/or remote operator) can send the information for the at least one image capturing device to the computer in or outside the suite. The computer can then relay this information to the at least one image capturing device or the computer can store this information and thereby control the at least one image capturing device. The cell therapy provider system (and/or remote operator) can update and/or maintain this information for the at least one image capturing device. For example, using the network connection, a cell therapy provider can push updates to the at least one image capturing device settings from the cell therapy provider system (and/or remote operator) to the computer (the computer can then send the information to the at least one image capturing device) and/or at least one image capturing device in or outside the cell therapy suite. This may be done either automatically or manually by personnel of the cell therapy provider. In response to receiving the information and/or in response to receiving a request from an operator, the at least one image capturing device can adjust its settings, capture images, and/or send additional information.

As explained above, the cell therapy suite can include computer 105. In some embodiments, computer 105 can be outside (e.g., remote) from a suite. In some embodiments, computer 105 can be the brain of the suite in that it can send information (e.g., commands) to any and all other components in the suite. In some embodiments, an operator can use the computer for patient onboarding. In some embodiments, the computer can be the main connection point to the cell therapy provider system (and/or remote operator). As such, any information sent from the cell therapy provider system (and/or remote operator) such as the library of cell therapy protocols to the cell therapy suite can be sent to the computer and then relayed to the other components in or outside the suite to perform the cell therapy treatment.

In some embodiments, the computer can have a display such as a graphical user interface for the operator to interact with the computer. In some embodiments, the computer can have display 118 described above. In some embodiments, a display may be separate from the computer. In addition, the display can provide the operator with instructions (which can be included in the library of cell therapy protocols) specific to any step in the cell therapy treatment process including instructions specific to the operating condition the operator has chosen for the cell-processing device. In some embodiments, the display can provide the operator with training instructions (which can be included in the library of cell therapy protocols) on how to operate any component of the cell therapy treatment process such as the various components in the suite.

In some embodiments, the computer can receive patient information either directly from an operator, from a component of the cell therapy system, or from the cell therapy provider system (and/or remote operator) (in the library of cell therapy protocols). In some embodiments, the computer (or display) can run an operator (e.g., technician, doctor, nurse, etc.) assistant or training program such as an augmented or virtual reality assistant or training program. In some embodiments, the library of cell therapy protocols can include the operator assistant or training program. For example, in response to receiving a request to train an operator of a cell therapy system, the system can perform a step of a respective cell therapy patient protocol from the library of cell therapy protocols (that includes the operator training program) that corresponds to the combination of the patient's indication and GOI, peptide, or small molecule. Thus, an operator can be trained on how to perform cell therapy for a combination of a specific patient's indication and GOI, peptide, or small molecule.

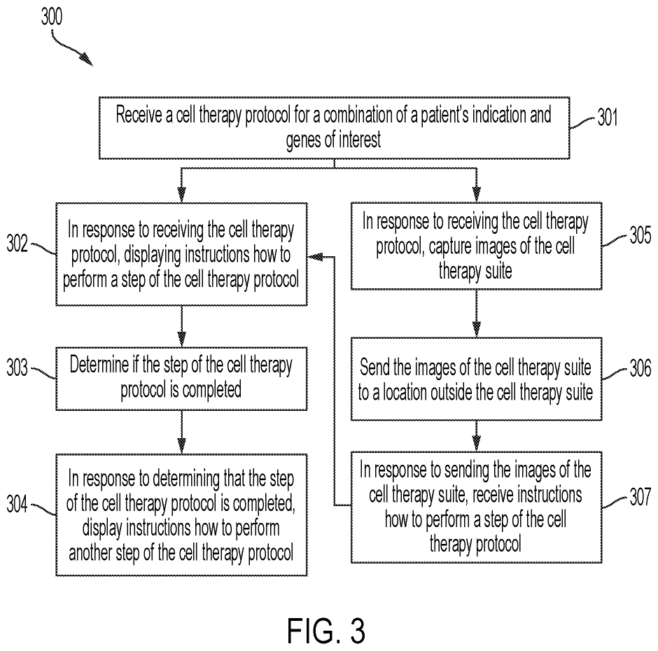

FIG. 3 depicts a flowchart representing an exemplary method 300 for providing cell therapy guidance, in accordance with some embodiments. As described in detail below, method 300 may enable a system (such as system 100) to receive a cell therapy protocol for a combination of a patient's indication and genes of interest (GOI), peptides, or small molecules; in response to receiving the cell therapy protocol, displaying instructions how to perform a step of the cell therapy protocol; determine if the step of the cell therapy protocol is complete; and in response to determining if the step of the cell therapy protocol is complete, displaying instructions how to perform another step of the cell therapy protocol. In addition, method 300 may also enable a system to receive a cell therapy protocol for a combination of a patient's indication and GOI, peptides, or small molecules; in response to receiving the cell therapy protocol, capture images of the cell therapy suite; send the images of the cell therapy suite to a location outside the cell therapy suite; and in response to sending the images of the cell therapy suite, receive instructions how to perform a step of the cell therapy protocol.

In some embodiments, method 300 may be carried out, in whole or in part, by one or more of the components of a system, such as system 100 described in FIG. 1 and the corresponding description. In some embodiments, any one or more of the aspects of method 300 may be combined, in whole or in part, with any one or more of the components of FIG. 1, and/or with any one or more of the systems, methods, devices, and/or techniques described elsewhere herein.

At block 301, in some embodiments, the system may receive a cell therapy protocol for a combination of a patient's indication and GOI, peptides, or small molecules. In some embodiments, the cell therapy protocol may be a cell therapy protocol from the library of cell therapy protocols. In some embodiments, the cell therapy protocol may not be a cell therapy protocol from the library of cell therapy protocols. In some embodiments, the system may receive a cell therapy protocol in response to a request for treating a patient with cell therapy and/or a request for training an operator to use a cell therapy suite. In some embodiments, the system may receive a cell therapy protocol in response to a request for treating a patient with cell therapy and/or a request for training an operator to use a cell therapy suite by remote monitor (e.g., supervisor of the operator) or personnel of the cell therapy provider. At block 302, in some embodiments, in response to receiving the cell therapy protocol, the system can display instructions how to perform a step of the cell therapy protocol. The instructions how to perform a step of the cell therapy protocol can include instructions how to operate any component of a cell therapy suite disclosed herein and/or what actions to take with respect to a patient. As explained above, the system may include at least one display. In some embodiments, the system includes a heads-up-display (HUD), wherein the instructions how to perform the first step of the cell therapy protocol are displayed on the HUD.

In some embodiments, the cell therapy protocol can include an operator assistant or training program. In some embodiments, these operator assistant or training programs can provide training programs/execution programs to operators in or outside the suite step-by-step instructions on how to perform cell therapy on a given patient including how to operate the various components in the suite and what actions to take with respect to the patient. In some embodiments, these programs can be run on a heads-up display (HUD) that an operator can wear as they perform cell therapy treatment for a patient or as they learn how to perform cell therapy treatment for a given patient as well as example videos that play per step.

In some embodiments, in response to receiving the cell therapy protocol, the system can provide auditory instructions how to perform a step of the cell therapy protocol. For example, in some embodiments, the display(s) can include a speaker that can provide the auditory instructions (e.g., audio cues) or other audio-related instructions (e.g., a remote supervisor can talk to the operator using the speaker).

At block 305, in some embodiments, in response to receiving the cell therapy protocol, the system can capture images of the cell therapy suite and/or patient. In some embodiments, the HUD can have at least one image capturing device (e.g., a front-facing camera (if not more for peripheral and other vision)), as previously described. In some embodiments, even operators unfamiliar with cell therapy procedures can use the augmented reality program with overlays (e.g., color highlighted overlays) and step-by-step instructions to operate the entire suite for the cell therapy procedure.

At block 306, in some embodiments, the system can send images of the cell therapy suite and/or patient to a location outside the cell therapy suite (e.g., a cell therapy provider system). For example, the system may send images of the cell therapy suite and/or patient to the cell therapy provider system. In some embodiments, the location outside the cell therapy suite can be the display of a remote operator (e.g., a supervisor) or personnel of the cell therapy provider. In some embodiments, the images can be a video stream. In some embodiments, the images can be stored in any component of the systems disclosed herein including the cell therapy provider system (and/or remote operator). In some embodiments, the images can include everything that an operator would be viewing through their HUD including any alerts, instructions, patient data, and/or suite data. In some embodiments, the images can be used for facial recognition to confirm a patient identification. In some embodiments, the remote operator can confirm patient identification. In some embodiments, the facial recognition of the patient identification is confirmed by a machine learning or AI-assisted algorithms.

At block 307, in some embodiments, in response to sending the images of the cell therapy suite (or patient), the system can receive instructions how to perform a step of the cell therapy protocol. In some embodiments, in response to receiving instructions how to perform a step of a cell therapy protocol, the system can display the instructions how to perform the step of the cell therapy protocol. In some embodiments, as explained above the HUD can include at least one front-facing camera. In some embodiments, the cell therapy suite can synchronize with the HUD to begin real-time monitoring and/or recording upon receiving a cell therapy protocol (or upon wearing the HUD). In some embodiments, the HUD can be a commercially available augmented reality or virtual reality glasses or goggles. In some embodiments, the at least one image capturing device can record the actions of the operator such that the operator can be monitored in real-time in order to assist, teach, or guide the operator in any portion of cell therapy treatment. In some embodiments, a remote operator (e.g., an operator who is not wearing the HUD) can double-check the operator's cell therapy treatment/training steps. In some embodiments, the instruction how to perform a step of a cell therapy protocol can come from the location outside the cell therapy suite such as by a remote operator (e.g., supervisor) or personnel of the cell therapy provider watching the video stream of an operator in a cell therapy suite. In some embodiments, the at least one image capturing device can record video for live or asynchronous remote assistance/training/monitoring. For example, in some embodiments, the instructions how to perform a step of a cell therapy protocol can be from a person (e.g., supervisor) viewing the images of the cell therapy suite (or patient) at the location outside the cell therapy suite. In some embodiments, the HUD has an audio and/or video call device such that the remote operator (e.g., supervisor) or personnel of the cell therapy provider can provide instructions to the operator via audio and/or video call. In some embodiments, these instructions can include technical assistance for operating a component of the cell therapy suite. In some embodiments, video can be streamed both ways to and from the HUD of the operator and the remote operator (e.g., supervisor) or personnel of the cell therapy provider. In addition, in some embodiments, the instructions can be streamed to the HUD of the operator. In some embodiments, the remote operator (e.g., supervisor) or personnel of the cell therapy provider can also use augmented reality to have telepresence and/or experience the cell therapy treatment through the eyes of the HUD wearing operator. In some embodiments, the instructions how to perform a step of a cell therapy protocol can include example videos of performing the step. The at least one display can display the example videos of performing this step.

In some embodiments, the HUD can be an augmented reality HUD, wherein the instructions how to perform a step of the cell therapy protocol are overlayed onto an actual component of the cell therapy suite. In some embodiments, the images taken by the at least one image capturing device can be sent to a processor of any component disclosed herein (including the cell therapy provider system) for processing in order to highlight, indicate, or identify a component of the cell therapy suite. For example, for a step of a cell therapy protocol, a HUD can display an outline around a component or components of the system corresponding to the step of the cell therapy protocol or cause a component of the system to glow or be highlighted such that it signals to the operator that that component is to be used for the step of the cell therapy protocol. Such highlighting, indication, or identification can be accomplished by computer algorithms such as AI-assisted or machine learning algorithms. For example, the images taken by the at least one image capturing device can be sent to a processor of any component disclosed herein (including the cell therapy provider system (and/or remote operator)) and compared with common and trained image sets to highlight, indicate, or identify components per-step and/or indicate errors.

For example, in some embodiments, the system can highlight, indicate, or identify a component of the cell therapy suite that an operator should use for performing a step of the cell therapy protocol. For example, the HUD can highlight a cell-processing device in a cell therapy suite for performing a step of the cell-processing of a cell therapy protocol. In some embodiments, the images of the cell therapy suite can include an image of a component identifier of a component of the cell therapy suite. These component identifiers can be on a component of the cell therapy suite. In some embodiments, the instructions how to perform a step of the cell therapy protocol can include instructions how to operate a component of the cell therapy suite. In some embodiments, the component identifier can be a QR code or a barcode corresponding to the component of the cell therapy suite. Accordingly, taking an image of a component identifier can cause the system to send the identity of the component that corresponds to the component identifier to the cell therapy suite (e.g., the display). In some embodiments, the captured images can be recorded for batch and/or treatment records. In some embodiments, the recorded images can be stored at any component disclosed herein (including the cell therapy provider system).

In some embodiments, augmented or virtual reality can be used to create a simulated cell therapy suite for a virtual simulated cell therapy training (e.g., gesture-based simulation training) or simulated/virtual cell therapy protocol. In some embodiments, the HUD can be a virtual reality HUD, wherein the instructions how to perform a step of the cell therapy protocol includes a virtual or simulated cell therapy suite. In other words, in some embodiments, operators unfamiliar with cell therapy procedures or those who do not have access to a cell therapy suite can use virtual reality programs that can create a virtual or simulated cell therapy suite and step-by-step instructions to operate the entire virtual suite for the cell therapy procedure. In some embodiments, the HUD can match a real treatment HUD, but all treatment-related visual and audio aspects (e.g., environment, patient, materials, components) can be simulated computer graphics. In some embodiments, the operator can train or be approved, certified, and/or registered for performing cell therapy in a suite without even being in a cell therapy suite. All components of a cell therapy suite can be simulated such that an operator in training can receive instructions how to perform a cell therapy protocol as if they were actually in a cell therapy suite. In some embodiments, the operator can simulate performing steps of a cell therapy protocol in the virtual or simulated cell therapy suite by performing gestures (either by hand or other input device such as a controller(s)) that relate to what the actual action or operation would be if the operator were in a real cell therapy suite. In some embodiments, proper completion records of the training steps, metrics, and/or data can be monitored and/or recorded.

In some embodiments, any component disclosed herein (including the cell therapy provider system (and/or remote operator)) can include a database of operators (e.g., registered operators) who have completed cell therapy training and/or potential operators. In some embodiments, this database can include treatments that each operator is trained on, the number of operations performed by each operator, and/or any ratings, feedback, and/or reviews of an operator's performance (e.g., provided by a supervisor), among others. In some embodiments, the database can include ratings and/or feedback for a given operator. In some embodiments, the ratings, feedback, and/or reviews can be provided by the personnel of the cell therapy provider, operator, any supervisor, or any patient of the operator. In some embodiments, the database can be stored and/or maintained at any component disclosed herein (including the cell therapy provider system (and/or remote operator)). In some embodiments, the database can be accessible through the internet/web, a smartphone (e.g., an application for a smartphone), any display (including the HUD (AR and VR) displays disclosed herein) of cell therapy system (both inside the cell therapy suite and those located outside the cell therapy suite). In some embodiments, personnel of the cell therapy provider, operators, supervisors, and/or patients can access the database. In some embodiments, personnel of the cell therapy provider, operators, supervisors, or patients can reject or veto or accept procedures and/or operators through the database.

At block 303, in some embodiments, the system can determine if a step of a cell therapy protocol is complete. In some embodiments, determining if a step of a cell therapy protocol is complete includes receiving an operator input indicating that the step of the cell therapy protocol is complete. In some embodiments, the system can display a digitized checklist for a step or steps of a cell therapy protocol. In some embodiments, as the operator completes a step, he can provide input (e.g., click a digital box, perform a gesture, say a verbal command, or just perform the step itself) indicating that the step is complete. In some embodiments, determining if a step of the cell therapy protocol is complete includes determining if the operator performed a gesture corresponding to a step of the cell therapy protocol. This can hold true for both an operator in a cell therapy suite and for an operator in a virtual reality training environment. For example, in a virtual reality training environment, the HUD may be displaying that the operator should turn on a component of the simulated cell therapy suite. In response to performing a gesture corresponding to turning on a component of the simulated cell therapy suite, the system would determine that the step of the cell therapy protocol was complete. In some embodiments, determining if a step of a cell therapy protocol is complete can include a remote operator (e.g., supervisor) or personnel of the cell therapy provider determining if the step of the cell therapy protocol is complete. In some embodiments, the system can automatically determine that a step of a cell therapy protocol was completed using the images captured by the image capture device. For example, the system may be able to compare images received from the image capture device with those of common and/or trained image sets to determine if the step of the cell therapy protocol was completed. In some embodiments, determining if a step of a cell therapy protocol is complete can be determined from feedback from one or more of the components of the system. In some embodiments, the feedback can be a change of parameter of one or more of the components. For example, a sensor of one of the components of the system can detect a change in a parameter (e.g., change of weight for a storage rack, refrigerator, etc. or change in electrical conduction) that can be based on a change or operation that an operator performed. The component(s) that experienced the change in parameter can communicate with the system to let the system know that a step of a cell therapy protocol that corresponds to that change of parameter was completed.

In some embodiments, in response to determining that a step of a cell therapy protocol is complete, the system (e.g., display, computer, controllers) can provide visual, auditory, or haptic feedback.

At block 304, in some embodiments, in response to determining that a step of a cell therapy protocol is complete, the system can display instructions how to perform another step of the cell therapy protocol. In some embodiments, the another step can be a subsequent step of the cell therapy protocol or the step that comes after the previous completed step.

The cell therapy provider system can update and/or maintain the various programs to help the operator perform or learn a given cell therapy procedure. For example, using the network connection, a cell therapy provider can push updates to the computer programs (which can be in the library of cell therapy protocols) from the cell therapy provider system (and/or remote operator) to a computer or display in or outside the cell therapy suite. This may be done either automatically or manually by personnel of the cell therapy provider. In some embodiments, the updates may be for a modification of the existing computer programs or for addition of a new computer program for cell therapy treatment or training. In some embodiments, the augmented reality program running in a headset or other device can communicate with other components (e.g., cell-processing device, computer, etc.) to properly display real-time information for the steps of the cell therapy process.

In some embodiments, augmented or virtual reality can be used in the actual cell therapy suite for physical training or monitoring. The use of augmented reality or virtual reality can potentially reduce regulatory requirement and/or reduce human error issues associated with cell therapy. Augmented or virtual reality can improve workforce/operator training and/or improve onboarding speed and efficiency. In some embodiments, augmented or virtual reality can reduce human error.

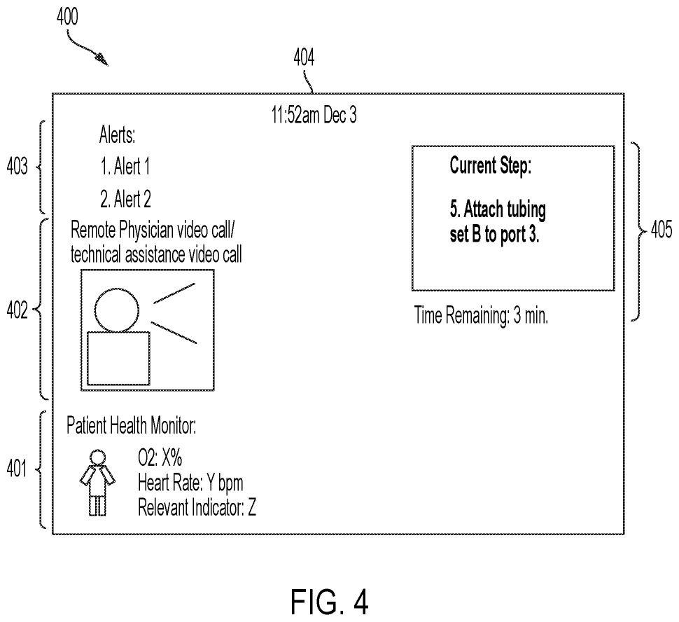

In some embodiments, any data, instructions, and/or other types of information can be displayed on at least one display of the system. For example, in some embodiments, the at least one display can display environment monitoring data from a cell therapy suite, patient health monitoring, and/or alerts for emergencies regarding the patient and/or cell therapy protocol. FIG. 4 illustrates an example of an augmented reality or virtual reality HUD 400 in accordance with some embodiments disclosed herein. As shown in FIG. 4, the HUD can display patient health data 401. In some embodiments, the patient health data can be from any component disclosed herein (including the cell therapy provider system) that is measuring data on the patient's health. In some embodiments, the HUD can display a video call 402. In other words, the HUD can display a video of a remote operator (e.g., supervisor) or personnel of the cell therapy provider who may be monitoring/assisting an operator who is performing cell therapy treatment. In some embodiments, the video may be a video of how to perform a step of a cell therapy protocol. In some embodiments, the HUD can display any alerts 403 or emergencies regarding the patient and/or cell therapy protocol. In some embodiments, the HUD can display the current time 404. In some embodiments, the HUD can display at least one step (e.g., the current step) 405 of a cell therapy protocol. In some embodiments, the HUD can display an estimated time remaining for completing the at least one step (e.g., the current step).

In order to expand and accelerate access to cell therapy to the general public, a provider of cell therapy services can install various versions of the cell therapy suites described herein in various locations including in a hospital depending on the need of a specific hospital. In some embodiments, the cell therapy provider can bear the cost partially or as a whole to install the suite in a location in order to relieve financial burden in areas where resources might not be allocable to cell therapy. For example, the cell therapy provider may enter into an agreement with another entity such as a hospital or medical treatment facility, city, State, etc. in which the provider chargers the entity a subscription fee for the cell therapy suite and/or fees for actual use of the cell therapy suite.

In some embodiments, such a subscription service can allow the entity to ask the cell therapy provider to create a c-srRNA for a particular indication. In turn, the cell therapy provider can find or select a proper GOI, peptide, or small molecule to treat the particular indication and produce a c-srRNA carrying the GOI, peptide, or small molecule. The production of the carrying vector (such as adeno-associated virus, DNA plasmid, RNA, self-replicating or self-amplifying RNA, or c-srRNA) can be performed in the cell therapy suite, provided by the cell therapy provider, or at a location outside of the suite.

By providing verification keys for subscription at the computer and/or display in or outside the suite, the cell therapy provider system (and/or remote operator) can verify that the operator can indeed use the suite as well as the various programs and instructions on how to provide cell therapy to a patient. For example, once a user logs into the computer in the suite, this suite computer can gain access to all the functions provided by the cell therapy provider system (and/or remote operator). Such relationship is a client-server configuration.

In addition, there may be no additional cost to subscribers of the cell therapy provider system (and/or remote operator) when adding new treatments or indications as any updates can be sent directly to the suite. The same holds true for any improvements to clinical protocols for cell therapy, changes required by regulatory body, or substitutions due to supply and logistics.

### Exemplary Process for an Operator Performing Cell Therapy in the Suite

The following is an exemplary process for providing cell therapy to a patient. First, the patient can arrive and be on-boarded by an operator (e.g., technician or physician). The operator may be using the AR device with HUD for steps/training/tracking procedure. Next, the operator can set up apheresis for patient while the patient is seated. The operator may receive patient data on the computer or AR device, and treat based on the data. In some embodiments, the apheresis device may receive information of which procedure patient is undergoing, and automatically obtain settings through the network. Apheresis can then be performed. Reagents, supplies can be taken from the storage rack, which may track usage, and the trash receptacle/biowaste receptacle holds the relevant trash. The apheresis machine may perform cell isolation by type, or the function can be done by the cell-processing device. In some embodiments, the patient can leave after receiving post-apheresis care from the operator. Next, the operator can attach reagents, tubing sets, etc. to the cell-processing device (potentially directed by augmented reality (AR)). The operator can then select the relevant program from a library on the cell-processing device or the computer, which can be updated in real time by the computer or the cell therapy provider system (and/or remote operator). The operator can perform the cell isolation, treatment of cells, which can all be done automatically by the cell processing machine while the operator changes connections and adds and removes reagents and bags. If treated cells need incubation or are being stored at a lower temperature, the incubator or refrigerator(s) and freezer can be used. Next, the patient can return which could be in 24 hours, or up to several weeks, all according to protocol. The operator then can set up infusion for patient and infuse the processed cells back into the patient. The operator then can perform care for the patient after infusion and the patient can leave while the operator cleans the suite and prepare for the next treatment.

### Exemplary Computing System

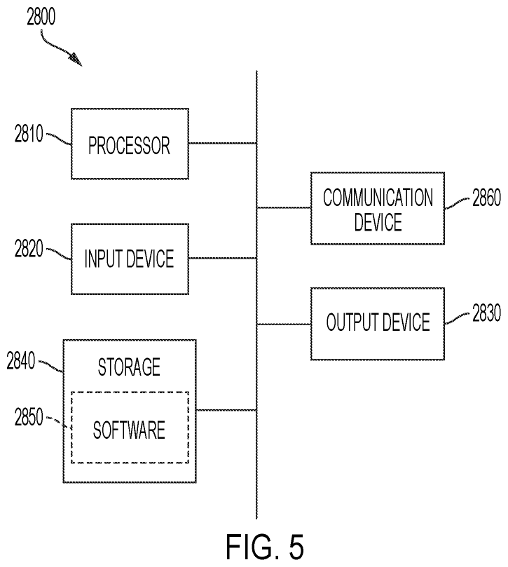

FIG. 5 illustrates an exemplary computing system, in accordance with some embodiments. Computing system 2800 can be a component of a system for cell therapy, for example system 100 and/or any of its subcomponents described above with respect to FIG. 1. For example, any and all of the components described in FIG. 1 may include their own computing system. In some embodiments, computing system 2800 may be configured to execute all or part of a method for providing cell therapy, such as all or part of methods 200 or 300 described above with respect to FIGS. 2 and 3.

Computing system 2800 can include a host computer connected to a network. Computing system 2800 can be a client computer or a server. As shown in FIG. 5, computing system 2800 can be any suitable type of microprocessor-based device, such as a personal computer; workstation; server; or handheld computing device, such as a phone or tablet. The computing system can include, for example, one or more of processor 2810, input device 2820, output device 2830, storage 2840, and communication device 2860.

Input device 2820 can be any suitable device that provides input, such as a touch screen or monitor, keyboard, mouse, or voice-recognition device. Output device 2830 can be any suitable device that provides output, such as a touch screen, monitor, printer, disk drive, or speaker.

Storage 2840 can be any suitable device that provides storage, such as an electrical, magnetic, or optical memory, including a RAM, cache, hard drive, CD-ROM drive, tape drive, or removable storage disk. Communication device 2860 can include any suitable device capable of transmitting and receiving signals over a network, such as a network interface chip or card. The components of the computer can be connected in any suitable manner, such as via a physical bus or wirelessly. Storage 2840 can be a non-transitory computer-readable storage medium comprising one or more programs, which, when executed by one or more processors, such as processor 2810, cause the one or more processors to execute methods described herein, such as all or part of methods 200 and 300 described above with respect to FIGS. 2 and 3.

Software 2850, which can be stored in storage 2840 and executed by processor 2810, can include, for example, the programming that embodies the functionality of the present disclosure (e.g., as embodied in the systems, computers, servers, and/or devices as described above). In some embodiments, software 2850 can be implemented and executed on a combination of servers such as application servers and database servers. The software may also include features of artificial intelligence or machine learning.

Software 2850 can also be stored and/or transported within any computer-readable storage medium for use by or in connection with an instruction execution system, apparatus, or device, such as those described above, that can fetch and execute instructions associated with the software from the instruction execution system, apparatus, or device. In the context of this disclosure, a computer-readable storage medium can be any medium, such as storage 2840, that can contain or store programming for use by or in connection with an instruction execution system, apparatus, or device.

Software 2850 can also be propagated within any transport medium for use by or in connection with an instruction execution system, apparatus, or device, such as those described above, that can fetch and execute instructions associated with the software from the instruction execution system, apparatus, or device. In the context of this disclosure, a transport medium can be any medium that can communicate, propagate, or transport programming for use by or in connection with an instruction execution system, apparatus, or device. The transport-readable medium can include, but is not limited to, an electronic, magnetic, optical, electromagnetic, or infrared wired or wireless propagation medium.

Computing system 2800 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Computing system 2800 can implement any operating system suitable for operating on the network. Software 2850 can be written in any suitable programming language or blockchain, such as C, C++, Java, or Python. In various embodiments, application software embodying the functionality of the present disclosure can be deployed in different configurations, such as in a client/server arrangement or through a Web browser as a Web-based application or Web service, for example.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Additionally, those skilled in the art would understand that one or more (e.g., all) of the above described components and features may be combined without departing from the scope of the disclosure. Finally, the entire disclosure of the patents and publications referred to in this application are hereby incorporated herein by reference.

1. A cell therapy suite comprising:
one or more processors configured to:
receive information comprising a library of cell therapy protocols for a plurality of combinations of patient indications and genes of interest (GOI);
receive a request for treating a patient with cell therapy based on a combination of the patient's indication and GOI; and
in response to receiving the request for treating a patient with cell therapy, perform a step of a respective cell therapy protocol from the library of cell therapy protocols that corresponds to the combination of the patient's indication and GOI.

2. The cell therapy suite of claim 1, wherein the received information is from a remote server location outside of the suite.

3. The cell therapy suite of claim 1, wherein the suite comprises a cell-processing device.

4. The cell therapy suite of claim 3, wherein the information comprising the library of cell therapy protocols comprises a plurality of operating conditions for the cell-processing device for the plurality of combinations of patient indications and GOIs.

5. The cell therapy suite of claim 4, wherein receiving a request for treating a patient with cell therapy based on a combination of the patient's indication and GOI comprises receiving a request for an operating condition of the cell-processing device based on a combination of the patient's indication and GOI.

6. The cell therapy suite of claim 5, wherein in response to receiving the request for the operating condition, the one or more processors are configured to process cells of the patient with RNA carrying the GOI in the cell-processing device according to a respective operating condition that corresponds to the combination of the patient's indication and GOI.

7. The cell therapy suite of claim 6, wherein the RNA is c-srRNA.

8. The cell therapy suite of claim 1, wherein the one or more processors are configured to receive modifications to the library of cell therapy protocols for a plurality of combinations of patient indications and GOIs.

9. The cell therapy suite of claim 1, wherein the cell therapy suite is a clean room that satisfies ISO 5, ISO 7, or ISO 8.

10. The cell therapy suite of claim 1, further comprising an RNA production unit configured to load GOIs onto an RNA vehicle.

11. The cell therapy suite of claim 10, wherein the one or more processors are configured to receive a patient's GOI identified by a sequencer and in response to receiving the patient's identified GOI, load the identified GOI onto an RNA vehicle in the RNA production unit or the one or more processors are configured to transmit the identified GOI information to a remote server location outside of the suite.

12. The cell therapy suite of claim 1, further comprising a sequencer to identify the patient's GOI.

13. The cell therapy suite of claim 1, further comprising an apheresis device for extracting the patient's cells.

14. (canceled)

15. (canceled)

16. (canceled)

17. (canceled)

18. (canceled)

19. (canceled)

20. (canceled)

21. (canceled)

22. (canceled)

23. A system for cell therapy treatment comprising:
one or more processors configured to:
receive a cell therapy protocol for a combination of a patient's indication and genes of interest (GOI); and
in response to receiving the cell therapy protocol, displaying instructions how to perform a first step of the cell therapy protocol; and
determine if the step of the cell therapy protocol is complete; and
in response to determining if the step of the cell therapy protocol is complete, displaying instructions how to perform a second step of the cell therapy protocol.

24. The system of claim 23, further comprising a heads-up-display (HUD), wherein the instructions how to perform the first step of the cell therapy protocol are displayed on the HUD.

25. The system of claim 24, wherein the HUD is an augmented reality HUD, wherein the instructions how to perform the first step of the cell therapy protocol are overlayed onto a component of a cell therapy suite.

26. The system of claim 25, wherein the instructions how to perform the first step of the cell therapy protocol identifies the component of the cell therapy suite to be used in the step of the cell therapy protocol.

27. The system of claim 24, wherein the HUD is a virtual reality HUD, wherein the instructions how to perform the first step of the cell therapy protocol comprises a simulated cell therapy suite.

28. The system of claim 24, wherein the HUD comprises an image capture device, and in response to receiving the cell therapy protocol, the image capture device captures images of a cell therapy suite.

29. The system of claim 23, wherein determining if the first step of the cell therapy protocol is complete comprises receiving a user input indicated the step of the cell therapy protocol is complete.

30. The system of claim 23, wherein determining if the first step of the cell therapy protocol is complete comprises determining if a user performs a gesture corresponding to the first step of the cell therapy protocol.

31. The system of claim 23, wherein in response to receiving the cell therapy protocol, providing auditory instructions how to perform the first step of the cell therapy protocol.

32. The system of claim 23, in response to determining if the step of the cell therapy protocol is complete, providing auditory or haptic feedback.

33. (canceled)

34. (canceled)

35. A system for cell therapy treatment comprising:
a cell therapy suite; and
one or more processors configured to:
receive a cell therapy protocol for a combination of a patient's indication and genes of interest (GOI);
in response to receiving the cell therapy protocol, capture images of the cell therapy suite and/or patient;
send the images of the cell therapy suite and/or patient to a location remote from the cell therapy suite; and
in response to sending the images of the cell therapy suite, receive instructions how to perform a step of the cell therapy protocol.

36. The system of claim 35, wherein the instructions how to perform the step of the cell therapy protocol are from the location outside the cell therapy suite.

37. The system of claim 36, wherein the instructions how to perform the step of the cell therapy protocol are from a remote operator viewing the images of the cell therapy suite and/or patient at the location outside the cell therapy suite.

38. The system of claim 35, wherein the images of the cell therapy suite comprise an image of a component identifier of a component of the cell therapy suite, wherein the instructions how to perform a step of the cell therapy protocol comprises instructions how to operate the component of the cell therapy suite.

39. The system of claim 38, wherein the component identifier is a QR code or a barcode corresponding to the component of the cell therapy suite.

40. The system of claim 35, wherein the images of the cell therapy suite and/or patient are video images of the cell therapy suite.

41. The system of claim 35, wherein the one or more processors are configured to display the instructions how to perform a step of the cell therapy protocol in response to receiving instructions how to perform the step of the cell therapy protocol.

42. The system of claim 41, further comprising a heads-up-display (HUD), wherein the instructions how to perform the first step of the cell therapy protocol are displayed on the HUD.

43. The system of claim 42, wherein the HUD is an augmented reality HUD, wherein the instructions how to perform the step of the cell therapy protocol are overlayed onto a component of a cell therapy suite.

44. (canceled)

45. (canceled)
